SEQUENCE LISTING (6 sequences)

SEQ ID NO: 1 - DNA, 49 bp, Artificial Sequence
  Primer mtDp1
aattctaatacgactcactatagggaagaaccgggctctgccatcttaa

SEQ ID NO: 2 - DNA, 20 bp, Artificial Sequence
  Primer mtDp2
gtaatccaggtcggtttcta

SEQ ID NO: 3 - DNA, 34 bp, Artificial Sequence
  Beacon mtDmb
cgtacgtgatatcatctcaacttagtatcgtacg

SEQ ID NO: 4 - DNA, 49 bp, Artificial Sequence
  Primer SnrpDp1
aattctaatacgactcactatagggagaggcccggcatgtggtgcataa

SEQ ID NO: 5 - DNA, 20 bp, Artificial Sequence
  Primer SnrpDp2
tgcgcctctttctgggtgtt

SEQ ID NO: 6 - DNA, 34 bp, Artificial Sequence
  Beacon SnrpDmb
cgcatgctgtaaccacgcactctcctcgcatgcg

(19) United States
(12) Patent Application Publication
(10) Pub. No.: US 2006/0172326 A1
(43) Pub. Date: Aug. 3, 2006

(54) HMG coenzyme a reductase inhibitors affect the mitochondrial DNA content of cells

(75) Inventors: Bob van Gemen, Almere (NL); Marinus Petrus de Baar, Amsterdam (NL)

Correspondence Address: TRASK BRITT, P.O. BOX 2550, SALT LAKE CITY, UT, 84110, US

(73) Assignee: PrimaGen Holding B.V., Amsterdam (NL)

(21) Appl. No.: 11/321,735

(22) Filed: Dec. 29, 2005

Related U.S. Application Data
(63) Continuation of application No. PCT/NL04/00468, filed on Jul. 1, 2004.

(30) Foreign Application Priority Data
Jul. 2, 2003 (EP) 03077075.4

(51) Int. Cl. C12Q 1/68

(52) U.S. Cl. 435/6

(57) ABSTRACT

Statins are widely used for the treatment of high cholesterol levels. In the present invention, the effect of statins on mitochondrial nucleic acid and the activation state of mitochondria is used in methods for determining whether a subject is at risk of developing side effects of statin treatment, methods for the treatment of a clinical symptom associated with reduced mitochondrial function. Further provided are kits and the like comprising a means for the detection of mitochondrial nucleic acid, or the activation of a mitochondrion for use in a method mentioned above.

### CROSS-REFERENCE TO RELATED APPLICATIONS

This application is a continuation of PCT International Patent Application No. PCT/NL2004/000468, filed on Jul. 1, 2004, designating the United States of America, and published, in English, as PCT International Publication No. WO 2005/003392 A2 on Jan. 13, 2005, which application claims priority to European Patent Application No. 03077075.4, filed Jul. 2, 2003, the contents of each of which are hereby incorporated herein by this reference.

### TECHNICAL FIELD

The invention relates generally to the field of medicine and biotechnology. More in particular, the invention relates to the field of diagnostics and pharmacology.

### BACKGROUND

HMG coenzyme A reductase inhibitors, also referred to as statins, are presently widely used to treat high cholesterol levels. Statins are very effective in getting the cholesterol level into the desired range, and they have had thus far few side effects. With their increased use and, in particular, with the recent clinical failure of cerivastatin, more of the side effects with prolonged use have become apparent.

In a minority of the patients, some side effects are detected after prolonged treatment with statins. Side effects are diverse and differ from patient to patient. Most prevalent are hepato-toxicity and myopathy. The grade of side effect may also vary. The observed toxicity is typically dose dependent and reversible upon cessation of treatment. Several risk factors have been identified that are associated with a higher prevalence of side effects. These risk factors vary but include patients with another disease and patients undergoing severe trauma or heavy surgery.

Statins act through a common pathway. They all inhibit the activity of HMG coenzyme A reductase. This enzyme is crucial in an early step of the cholesterol biosynthesis pathway. By inhibiting this biosynthesis, most, if not all, of the endogenous cholesterol syntheses capacity can be turned off, thereby contributing significantly to a general lower cholesterol level in the circulation. HMG coenzyme A reductase is a cytoplasmic enzyme that utilizes Acetyl-CoA as one of its substrates. Acetyl-CoA can be generated from various sources but is typically made available to the enzyme via the mitochondrion of the cell.

### SUMMARY OF THE INVENTION

It has been found that statins have a pronounced effect on the mitochondrial nucleic acid content of a cell. The cells of subjects are treated with statins in dose ranges that are suitable for the treatment of high cholesterol levels. As a result of this treatment, the mitochondrial nucleic acid content rises in the cell. Mitochondrial nucleic acid content is thus indicative of the use of a statin by a subject.

Thus, in one aspect, the invention provides a method for monitoring a subject receiving an HMG CoA reductase inhibitor, the method comprising determining in a sample of a subject a first parameter indicative for the mitochondrial nucleic acid content of the sample. The parameter may be used as an absolute value or may be related to, for instance, the sample volume obtained or another parameter capable of placing the first parameter in a context.

### DESCRIPTION OF THE DRAWING

FIG. 1. Median and interquartile mitochondrial DNA values in PBMC of patients treated with simvastatin (left panel) or fibrates (right panel). Compared were values measured at the start of the therapy (T=0) and after two months on the respective therapy (T=2 months). Clearly, simvastatin causes an increase in the number of mitochondrial DNA copies in PBMC compared to fibrates.

### DETAILED DESCRIPTION OF THE INVENTION

In a preferred embodiment, a second parameter indicative for the cellular content of the sample is determined. The two parameters can be related to one another to arrive at a parameter that reflects the relative mitochondrial nucleic acid content per cell in the sample. The mitochondrial nucleic acid content per cell ratio is a more accurate predictor of the action of statin in the body of the subject. The method may, of course, also be used in vitro, for instance, in cell lines. Some statins require metabolic conversion prior to becoming active as an HMG coenzyme A reductase. This metabolic conversion is typically done by tissue cells of the subject, particularly the liver. Thus, when the method is performed in vitro with statins requiring metabolic conversion, it is preferred to use the converted statin directly or make metabolic conversion available in the in vitro system. A person skilled in the art is able to design suitable tests for his/her purpose.

Thus, while a method of the invention is preferably performed with at least two samples, where at least one is obtained prior to initiation of treatment, the person skilled in the art is capable of adjusting the experimental design to his/her special needs. It is, for instance, possible to compare the mitochondrial nucleic acid with historical controls.

In a preferred embodiment, a value obtained for at least a first parameter indicative for the mitochondrial nucleic acid content of the sample is compared with a reference. The reference can be an historical control or a sample obtained prior to initiation of treatment. There are, however, many other particular designs possible that are also encompassed in the present invention.

The parameter for the mitochondrial nucleic acid content is preferably determined on the basis of mitochondrial DNA. Mitochondrial RNA can also be used. However, the parameter for the mitochondrial nucleic acid content may also be an indirect parameter, such as, for instance, the mitochondrial volume or activity in the cell. Particularly, the mitochondrial RNA or protein content is a parameter for the functionality and activity of the mitochondria and may thus also be used to monitor a subject receiving an HMG CoA reductase inhibitor.

Similarly, the parameter for the cellular content of the sample may be inferred through indirect parameters. However, preferably it is performed by means of determining the cellular nucleic acid content. The nucleic acid content of cell and/or mitochondrion can be determined in its entirety, thus, for instance, all mitochondrial nucleic acid may be determined or all nucleic acid of the cell compared with the mitochondrial nucleic acid content of the cell. As mentioned, many different parameters may be determined and used to set off the mitochondrial nucleic acid content of a sample.

In a preferred embodiment, the copy number of mitochondrial versus cellular DNA is determined. The copy number is an accurate measure of the mitochondrial nucleic acid of a cell. It is not necessary to determine this content in its entirety. One may measure just a part of the nucleic acid in the mitochondrion or cell. As the cellular genome varies between 1 (diploid) copy to two (diploid) copies per cell, one may also estimate the relative number of copies of the genome based on a value for only part of the genome, say (a part of) a gene. Similarly, the mitochondrial genome is present in a certain number of copies a cell. Thus, not all mitochondrial DNA need be measured. It suffices to determine a parameter for this content based on only part of the mitochondrial genome, say a suitable gene. The same holds true when RNA is used to determine the parameter. A specific RNA or part thereof may be used as an indication for what occurs with the total RNA content of the cell or the mitochondrion. Many methods are suitable to measure mitochondrial nucleic acid versus cellular nucleic acid, or nuclear nucleic acid. For example, suitable methods for determining the ratio of mitochondrial versus cellular nucleic acid are described in PCT/NL01/00883 and PCT/US01/147223, the contents of which are incorporated by reference herein.

The method may be used to determine whether a subject is undergoing statin treatment. However, the method can also be used to monitor a subject receiving statin treatment. An increase in the mitochondrial nucleic acid content of a cell is indicative for the number and/or the activation of mitochondria in the cell. The more mitochondrial nucleic acid, the more mitochondrions are in the cell and/or the higher the activity is of the mitochondria in the cell. In the present invention, it was found that cells normally respond to statins in the environment by increasing the number of mitochondria and/or activating their mitochondria. Subjects at risk of developing side effects from the treatment with statins, however, respond in an abnormal manner, or are, at least in part, abnormal in responding in this manner. This abnormal behavior is apparent prior to clinical manifestation of side effects.

It is thus possible to determine if a subject is at risk of developing side effects from a statin treatment, by determining whether the mitochondrial nucleic acid content of a sample obtained from the subject displays the marked increase in response to the treatment within a normal range observed in individuals with effective statin treatment without side effects. If the increase is absent or not sufficiently high, the subject is at risk. Alternatively, the increase may be abnormally high, indicative for an abnormal high mitochondrial activity and/or volume, which can result in overheating of the mitochondria and subsequent loss of mitochondrial function and eventually mitochondrial death and lysis.

The method can be performed at any stage prior, during or after treatment of the subject with a statin. If done before, the method is preferably performed in vitro, as mentioned above. Thus, the invention further provides a method of the invention further comprising determining from the first and/or second parameter whether the subject is developing a side effect from the inhibitor, or is at risk of developing such a side effect. In cases where it is determined that the subject is at risk, the treatment of the subject may be altered as a result of the risk. The alteration may comprise additional treatment to, at least in part, prevent a side effect of the statin treatment. However, preferably, the alteration comprises the cessation of the statin treatment.

The sample may be obtained from any part of the body of the subject. As mentioned above, statins are very hepatotrophic. On the other hand, a major side effect of statin treatment occurs in the muscle tissue of the subject. The sample may thus comprise a liver or muscle sample. Samples may also be taken from other parts of the subject. Preferably, the sample comprises blood cells. The sample is preferably a cellular sample that can be obtained in a non-invasive manner. Blood samples are typically regarded as non-invasive samples and are for the present invention preferred. Non-invasive cellular samples can also be obtained from other parts of the body of a subject and include, but are not limited to, mucosal surface samples such as from the mouth. The mitochondrial nucleic acid content and/or cellular content are preferably determined in the peripheral blood mononuclear cell (PBMC) fraction of the blood sample. Such samples are routinely generated in a clinical lab.

A change of the mitochondrial nucleic acid content of a cell is, as mentioned above, indicative for a change in the activation state of the mitochondria in the cell. Thus, by providing a cell with a statin, it is possible to enhance mitochondrial function in the cell. This embodiment of the invention is useful for the treatment of subjects suffering from diseases related to, or associated with, a reduced mitochondrial function. Thus, the invention provides a method for, at least in part, reducing a clinical symptom related to a reduced mitochondrial function in a (part of a) subject, the method comprising administering an HMG CoA reductase inhibitor to the subject. Such clinical symptoms preferably comprise fatigue, (peripheral) neuropathy, (cardio) myopathy, lactic acidosis, liver failure, lipodistrophy, lipoatrophy, heart disease, hepatic steatosis, diabetes, Osteoarthritis, infertility problems, Parkinson's disease or viral infections like HIV-1, HCV, HBV.

It is also possible to treat subjects that are at risk of developing a symptom associated with or related to a reduced mitochondrial function. Statins are preferably used for the treatment of subjects undergoing treatment with a nucleotide analogue. Such analogues are typically used in the treatment of virus infection. The treatment is based on the inhibition of virus-replication through interference with nucleic acid polymerases specific for the virus. Such analogues have a common side effect that they also inhibit cellular polymerases including those polymerases specific for cellular organelles containing their own nucleic acid, such as mitochondria and chloroplasts. This inhibition of cellular polymerases is one of the causes of side effects in the treatment with nucleotide analogues.

The present invention provides a means to boost at least mitochondrial nucleic acid content of a cell and thereby counteracts the action of the nucleotide analogue, at least to some extent. The invention thus further provides a method for the treatment of a subject receiving a nucleotide analogue comprising providing the subject with a statin.

The statin is to be given in effective dosages in order to raise the mitochondrial nucleic acid content in a sample taken from the subject. Effective dosages vary from statin to statin and generally overlap the range effective in the reduction of cellular cholesterol synthesis in a subject. Statins that may be used for the present invention comprise Pravastatin (PRAVACHOL™), lovastatin (MEVACOR™), simvastatin (ZOCOR™), fluvastatin (LESCOL™), atorvastatin (LIPITOR™), cerivastatin (BAYCOL™), rosuvastatin (CRESTOR™) or lovastatin (ADVICOR™). The above-mentioned statins are preferred because they have already been on the market (with the exception of cerivastatin). However, it may be clear that any compound with HMG coenzyme A reductase inhibiting activity may be used in the present invention. Such compounds may be a functional part, derivative and/or analogue of a preferred statin as mentioned above. However, it may also be a different compound with the same HMG coenzyme A reductase activity in kind, not necessarily in amount. In a preferred embodiment, the statin comprises Simvastatin or a functional part, derivative and/or analogue thereof.

The invention is further described with the aid of the following illustrative Examples.

### EXAMPLES

### Example 1

Experiment: seven patients between 43 and 62 years old and all with elevated total cholesterol levels started using 20 mg Simvastatin per day for a period of two months to reduce their cholesterol levels. Three control patients with the same indications used fibric acid derivatives to reduce their cholesterol levels. In total, the study included x male and y female patients. At day 0 and after two months, whole blood samples were taken using CTP tubes (Beckton-Dickinson) and PBMC were purified out of these samples using standard protocols provided by the manufacturer of the tubes. The samples were checked for the necessary absence or low levels of thrombocytes (less than five per cell). All samples fulfilled these criteria. In these samples the mtDNA content per PBMC was determined using the Primagen Retina MiTox DNA assay.

Nucleic acids were isolated from 10⁵ PBMC according to the method described by Boom et al. and dissolved in 50 μl DNAse- and RNAse-free water. Five μl of the nucleic acid (equivalent to 1,000 PBMC) was put in the reaction mix to amplify the specific targets. In parallel, 10³ molecules of plasmid containing Snrp DNA was mixed with 4×10⁵, 2×10⁵, 10⁵, or 5×10⁴ molecules of plasmid containing mitochondrial DNA, and the mixture was used as input for the reactions. Reactions were performed in a 20 μl reaction volume and contained: 40 mM Tris-pH 8.5, 90 mM KCl, 12 mM MgCl2, 5 mM dithiotreitol, 1 mM dNTPs (each), 2 mM rNTPs (each), 0.2 μM primer (each), 0.05 μM molecular beacon, 1.5 units restriction enzyme Msp I, 375 mM sorbitol, 0.105 μg/μl bovine serum albumin, 9.6 units AMV RT, 64 units T7 RNA polymerase, 0.08 units RNAse H and input nucleic acid. The complete mixture, except the enzymes, sorbitol and bovine serum albumin was, prior to adding the enzyme mixture, incubated at 37° C. for 25 minutes and subsequently heated to 95° C. for two minutes in order to denature the DNA and to allow the primers to anneal. After cooling the mixture to 41° C., the enzyme mixture was added. The amplification took place at 41° C. for 90 minutes in a fluorimeter (Primagen Retina Reader) and the fluorescent signal was measured every minute (using the filter set 530/25 nm and 485/30 nm).

The reaction mix (duplex-mix) contained two sets of primers and beacon: SnrpD p1 and SnrpD p2 (for nuclear DNA, each 0.2 μM), and MtD p1 and MtD p2 (for mitochondrial DNA, each 0.2 μM) with beacons SnrpD mb (ROX-labeled) and MtD mb (FAM-labeled) (each 0.04 μM).

In a duplex reaction with two competing amplifications, the ratio of the slope of the curves of fluorescence in time is proportional to the ratio of the amount of molecules of each amplified species. The data of the plasmid Snrp/mitochondrial DNA mixtures were used to create a standard curve on which the unknown ratio of mitochondrial to Snrp nuclear DNA of the PBMC samples could be assessed.

| | |
|---|---|
| + | |
| Name Sequence 1 | |
| MtD p1 | |
| 5′ AAT TCT AAT ACG ACT CAC TAT AGG | (SEQ ID NO: _) |
| GAA GAA CCG GGC TCT GCC ATC TTA A 3′ | |
| MtD p2 | |
| 5′ GTA ATC CAG GTC GGT TTC TA 3′ | (SEQ ID NO: _) |
| MtD mb | |
| 5′ CGT ACG TGA TAT CAT CTC AAC TTA | |
| GTA TCG TAC G | |
| SnrpD p1 | |
| 5′ AAT TCT AAT ACG ACT CAC TAT AGG | (SEQ ID NO: _) |
| GAG AGG CCC GGC ATG TGG TGC ATA A 3′ | |
| SnrpD p2 | |
| 5′ TGC GCC TCT TTC TGG GTG TT 3′ | (SEQ ID NO: _) |
| SnrpD mb | |
| 5′ CGC ATG CTG TAA CCA CGC ACT CTC | (SEQ ID NO: _) |
| CTC GCA TGC G 3′ | |

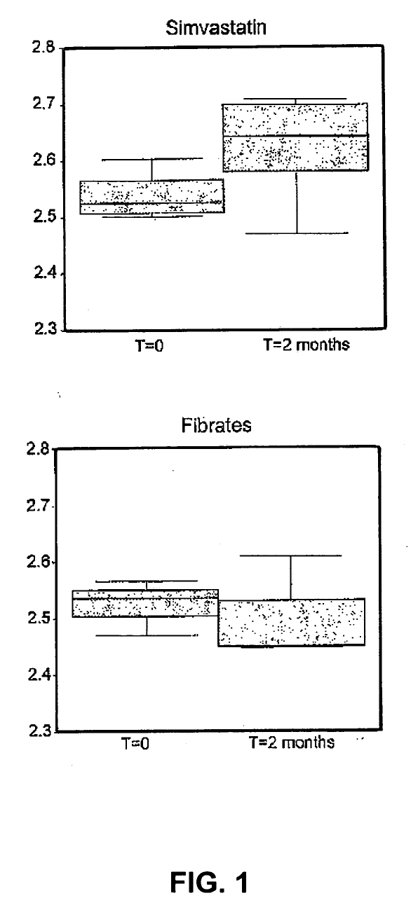

The results (see FIG. 1) were analyzed using the paired samples T-test as implemented in SPSS for Windows, version 11.5.1 (SPSS Inc., Chicago, Ill., U.S.A.). Normal distribution of mtDNA data is obtained after a 10th base logarithm transformation of mtDNA copies per cell. The p-value resulting from the paired samples T-test on the logarithmically transformed data was 0.045 with higher mtDNA values after two months relative to baseline values. This indicates a significantly increasing mtDNA content due to statin use. No changes were observed in the control group of three patients treated with fibric acid derivatives (fibrate), (p=0.737). In this latter group, there could be more a trend towards decrease of mtDNA content, but to state this, larger groups should be treated and analyzed.

We also observed a trend towards increase in mtRNA due to the use of statins, but this trend was not as strong as observed for mtDNA.

Simvastatin is a potent inhibitor of HMG-CoA reductase, thereby inhibiting the synthesis of cholesterol, but also intermediate products and products that use cholesterol as base, or share parts of the biosynthesis pathway like Co-enzyme Q10. Co-enzyme Q10 is involved in the oxidative respiratory chain in the mitochondrion. Since the mitochondria are involved in the synthesis of part of the proteins involved in the oxidative respiratory chain, increased levels of mtDNA as observed in the studied individuals suggest that the cell is compensating its loss in oxidative capacity and probably also other functions by increased synthesis of both mtDNA and so probably its functional proteins.

1. A method for monitoring a subject receiving an HMG CoA reductase inhibitor, said method comprising:
determining in a sample from the subject a first parameter indicative of the mitochondrial nucleic acid content of said sample.

2. The method according to claim 1, further comprising determining in said sample a second parameter indicative for cellular content.

3. The method according to claim 1, wherein at least one of said first and second parameters is compared with a reference.

4. The method according to claim 1, further comprising determining from said first and/or second parameter whether the subject is developing a side effect from said HMG CoA reductase inhibitor, or is at risk of developing such a side effect.

5. The method according to claim 1, further comprising altering the treatment of the subject in view of the monitoring.

6. A method for at least in part reducing a clinical symptom related with a reduced mitochondrial function in a subject or part of a subject, said method comprising:
administering an HMG CoA reductase inhibitor to a subject suffering from said clinical symptom.

7. The method according to claim 6, wherein the subject is suffering from or at risk of suffering from fatigue, neuropathy, peripheral neuropathy, myopathy, cardio myopathy, lactic acidosis, liver failure, lipodistrophy, lipoatrophy, heart disease, hepatic steatosis, diabetes, Osteoarthritis, infertility problems, Parkinson's disease or a viral infection.

8. The method according to claim 7, wherein said viral infection is HIV-1, HCV or HBV.

9. The method according to claim 6, wherein the subject is being treated with a nucleotide analogue.

10. The method according to claim 7, wherein the subject is being treated with a nucleotide analogue.

11. The method according to claim 8, wherein the subject is being treated with a nucleotide analogue.

12. A method of treating mitochondrial dysfunction in a subject believed to be in need thereof, said method comprising:
administering to the subject an HMG CoA reductase inhibitor so as to treat mitochondrial dysfunction in the subject.

13. A method for at least in part improving mitochondrial function in a cell culture, said method comprising:
contacting said cell culture with an HMG CoA reductase inhibitor so as to at least in part improve mitochondrial function in the cell culture.

14. The method according to claim 11, wherein said cell is cultured in vitro.

15. A method for determining whether a subject is at risk of suffering from a side effect of a treatment with an HMG CoA reductase inhibitor, said method comprising:
determining in a cellular sample from the subject whether mitochondrial nucleic acid is affected in response to in vitro exposure to said HMG reductase inhibitor.

16. The method according to claim 2, further comprising determining from said first and/or second parameter whether the subject is developing a side effect from said HMG CoA reductase inhibitor, or is at risk of developing such a side effect.

17. The method according to claim 3, further comprising determining from said first and/or second parameter whether the subject is developing a side effect from said HMG CoA reductase inhibitor, or is at risk of developing such a side effect.

18. The method according to claim 2, further comprising altering the treatment of the subject in view of the monitoring.

19. The method according to claim 3, further comprising altering the treatment of the subject in view of the monitoring.

20. The method according to claim 4, further comprising altering the treatment of the subject in view of the monitoring.
